(19) United States
(12) Patent Application Publication
(10) Pub. No.: US 2005/0051178 A1
(43) Pub. Date: Mar. 10, 2005

(54) Mouthguard for medical procedures

(76) Inventor: Michael David Sawford, Essex (GB)

Correspondence Address: SMITH, GAMBRELL & RUSSELL, LLP, 1850 M STREET, N.W., SUITE 800, WASHINGTON, DC, 20036, US

(21) Appl. No.: 10/909,366

(22) Filed: Aug. 3, 2004

(30) Foreign Application Priority Data
Sep. 4, 2003 (GB) 0320737.0

(51) Int. Cl. A61F 5/56

(52) U.S. Cl. 128/848

(57) ABSTRACT

A mouthguard (10) is described for use by a patient undergoing a medical procedure which involves passing an endoscope or similar instrument into the patient's mouth. The mouthguard (10) comprises a rigid tubular mouthpiece (12) formed integrally with a flexible restraining strap (14) which is dimensioned to encircle a patient's head. Fastening means (22, 24) are provided at one end of the strap (14) which can be fitted over one of a plurality of openings (26) in the other end of the strap (14) to secure the device in place comfortably.

The present invention relates to a mouthguard for use by a patient undergoing a medical procedure involving an endoscope or similar instrument that must be passed into the patient's mouth.

An endoscopic examination in which an endoscope is passed into a patient's mouth and down the throat is not a particularly pleasant procedure for the patient to undergo, and the patient may have a tendency to bite down to the medical instrument as it passes through the mouth, inhibiting the procedure or damaging the instrument.

The present invention provides a mouthguard device for use in medical procedures, comprising a rigid tubular mouthpiece to be received in a patient's mouth, a restraining member integral with the mouthpiece and comprising a flexible strap dimensioned to encircle a patient's head, and fastening means to secure the strap and mouthpiece in position.

Thus, the invention improves patient comfort by providing a mouthpiece which can be securely but comfortably held in position, giving the patient something to bite down on, whilst providing an incompressible aperture through which the medical instrument can be inserted to facilitate carrying out of the procedure.

Preferably, the fastening means is adjustable, so that the strap can be secured in different positions to accommodate different patients with maximum comfort.

The fastening means may typically comprise a projection formed at one end of the device and a series of apertures formed in the opposing end of the device whereby in use the projection can be passed through an appropriate one of the apertures to secure the device in position.

The strap may be narrowed in the vicinity of the aperture, to increase flexibility and assist in fastening the device.

Preferably, the strap is provided with one or more elongate slots therethrough to provide additional flexibility and increase patient comfort as the device is fitted.

The tubular mouthpiece is preferably provided with an outer elastomeric layer, to provide a soft surface into which the patient can bite.

The tubular mouthpiece preferably has a oval or elliptical cross-section to most comfortably fit within a patient's mouth.

The strap may be widened around the mouthpiece so as to substantially cover a patient's lips in use.

One or more additional openings may be provided in the strap adjacent the mouthpiece to allow access to the patient's mouth in use.

The invention will now be described in detail, by way of example only, with reference to the accompanying drawings in which:

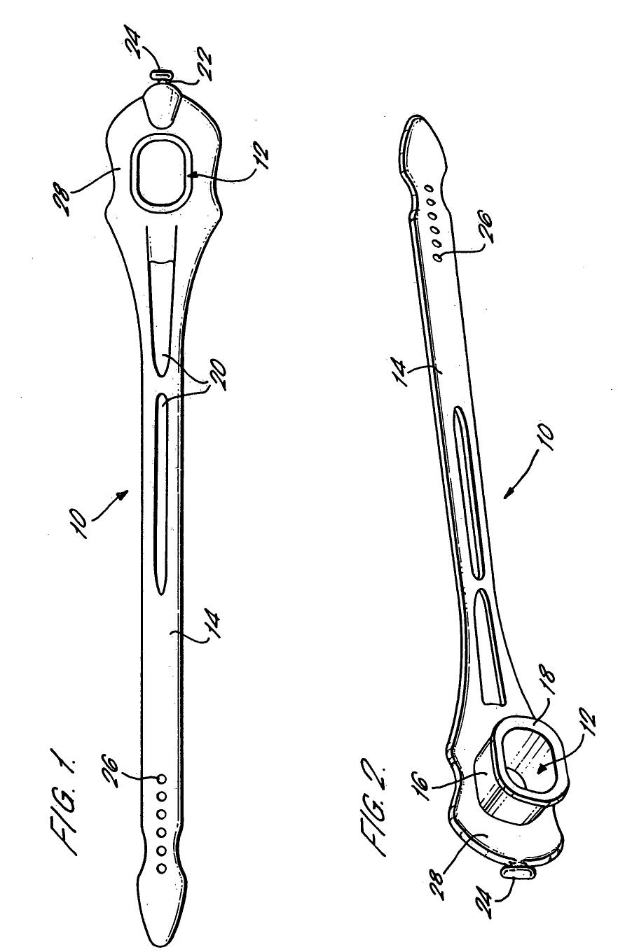

FIG. 1 is a front view of one embodiment of the present invention;

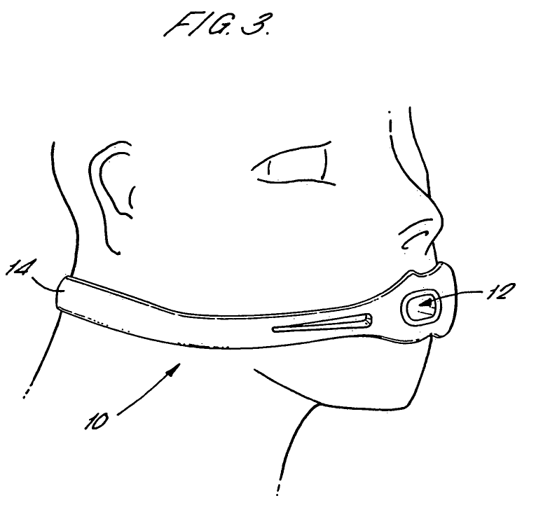

FIG. 2 is an enlarged perspective view of the rear of the device of FIG. 1;

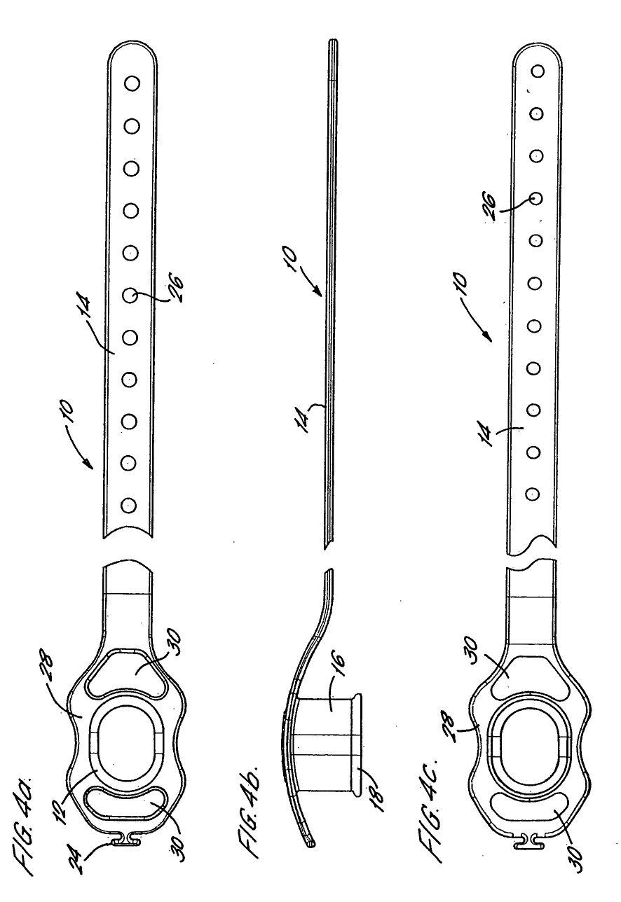

FIG. 3 is a perspective view of the device of FIGS. 1 and 2 in use on a patient; and

FIGS. 4a, 4b and 4c are front, side and rear views of another embodiment of the present invention.

As shown in FIGS. 1 and 2, the mouthguard device 10 comprises a tubular mouthpiece 12 with an integral restraining strap 14. The mouthpiece 12 consists of a short length of tube 16 preferably formed with a rigid polymer core and an elastomeric outer layer. At its proximal end, the tube 16 is integrally formed with the strap 14 as described further below. At its distal end, the tube 16 is flared outwardly to provide a lip 18. The tube 16 is sized to be comfortably received in a patient's mouth, with the elastomeric outer layer providing a soft texture that the patient can bite down on, whilst the rigid core prevents the patient biting down onto the medical instrument itself which is passed through the tube 16. The rigid core also provides structural rigidity to the whole device 10 and a solid fixing point for the strap 14.

The tube 16 is preferably oval or elliptical in cross-section, being wider than it is high, again to more comfortably fit in a patient's mouth. The lip 18 assists with retaining the mouthpiece 12 in the patient's mouth and resists accidental dislodgement, for example as the endoscope is being withdrawn.

The restraining strap 14 is formed from an elastomeric material integrally moulded with the mouthpiece 12. The strap 14 is sufficiently flexible to allow it to be fitted around a patient's head and secured onto itself as further described below, but not so flexible that accidental pulling, for example on the mouthpiece 12 as an endoscope is manipulated, could pull the strap 14 away from the patient's face and risk it snapping back painfully against the patient.

The strap 14 may be formed with one or more elongate slots 20 to give some additional flexibility and comfort when fitting the strap around a patient's head.

The strap 14 is also provided with adjustable securement means to secure the device 10 onto a variety of patients with differing head dimensions. In this example, the first end of the strap 14 adjacent to the mouthpiece 12 is formed with a projecting lug 22 having an enlarged head 24. The other end of the strap 14 remote from the mouthpiece 12 is formed with a series of apertures 26.

To secure the device 10 on a patient, the mouthpiece 12 is inserted into the mouth and the strap 14 passed around the back of the head as seen in FIG. 3. The lug 22 is then passed through an appropriate one of the apertures 26 to secure the device 10 in position. The strap 14 may be slightly narrowed in the vicinity of the apertures 26 to give slightly increased flexibility at that point, to assist with attaching and detaching the strap 14 over the lug 22.

The strap 14 may be widened around the mouthpiece 12 so as to form a faceplate 28 which will substantially cover a patient's lips, for protection and comfort during the procedure.

By way of example only, the length of the strap 14 from the lug 22 to the opposing end may be approximately 610 mm, with a maximum width of approximately 40 mm, and the mouthpiece may have a length of approximately 27.5 mm. The rigid core of the mouthpiece 12 may be formed of silicon and the elastomeric layer and the strap 14 may be food grade polypropylene. The device 10 may be manufactured by means of a over moulding or two shot moulding process.

A second embodiment of the present invention is shown in FIGS. 4a to 4c using the same reference numerals where appropriate. The endoscope mouthguard device 10 of this embodiment is generally similar to the first embodiment, but in this case additional openings 30 are provided in the faceplate 28 adjacent, in this case to either side of, the mouthpiece 12. These openings 30 provide an additional access to the patient's mouth for the purpose of aspiration in the event of the main lumen becoming obstructed during a procedure.

Thus, the present invention provides a mouthguard device which is easily fitted and provides maximum comfort and protection for a patient undergoing an endoscopic procedure, whilst also protecting a medical instrument itself and facilitating manipulation of the instrument to perform the procedure most effectively. It will be appreciated that a number of variations and modifications to the precise details described above may be made without departing from the scope of the invention as set out in the claims.

1. A mouthguard device for use in medical procedures, comprising a rigid tubular mouthpiece to be received in a patient's mouth, a restraining member integral with the mouthpiece and comprising a flexible strap dimensioned to encircle a patient's head, and fastening means to secure the strap and mouthpiece in position.

2. A mouthguard device as claimed in claim 1, wherein the fastening means is adjustable.

3. A mouthguard device as claimed in claim 2, wherein the fastening means comprises a projection formed at one end of the device and a series of apertures formed in the opposing end of the device, whereby in use the projection can be passed through an appropriate one of the apertures to secure the device in position.

4. A mouthguard device as claimed in claim 3, wherein the strap is narrowed in the vicinity of the apertures.

5. A mouthguard device as claimed as claimed in claim 1, further comprising one or more elongate slots formed in the flexible strap.

6. A mouthguard device as claimed in claim 1, further comprising an elastomeric outer layer formed on the tubular mouthpiece.

7. A mouthguard device as claimed in claim 1, wherein the tubular mouthpiece is oval or elliptical in cross-section.

8. A mouthguard device as claimed in claim 1, wherein the strap is widened around the mouthpiece so as to substantially cover a patient's lips in use.

9. A mouthguard device as claimed in claim 1, further comprising at least one additional opening in the strap adjacent the mouthpiece to allow access to the patient's mouth in use.
